(19) United States
(12) Patent Application Publication
(10) Pub. No.: US 2026/0209153 A1
(43) Pub. Date: Jul. 23, 2026

(54) PROCESS FOR THE SYNTHESIS OF ISOPHORONE IN THE LIQUID PHASE WITH RECYCLING OF BY-PRODUCTS

(71) Applicant: ARKEMA FRANCE, Puteaux (FR)

(72) Inventor: Christophe RUPPIN, La Chambre (FR)

(73) Assignee: ARKEMA FRANCE, Puteaux (FR)

(21) Appl. No.: 19/138,022

(22) PCT Filed: Dec. 18, 2023
(86) PCT No.: PCT/EP2023/086416

§ 371 Date: Jun. 11, 2025

(30) Foreign Application Priority Data
Dec. 19, 2022 (FR) FR2213776

(51) Int. Cl. C07C 45/74; C07C 45/82

(52) U.S. Cl. CPC C07C 45/74; C07C 45/82; C07C 2601/16

(57) ABSTRACT

The present invention relates to a continuous process for the liquid-phase synthesis of isophorone by alkaline self-condensation of acetone, comprising the following successive steps:
a) continuous injection through a tubular reactor (R) of a stream of an aqueous alkali metal hydroxide solution and of a stream of an organic solution comprising acetone and by-products recycled via step g), followed by
b) condensation reaction of acetone within the tubular reactor (R), followed by
c) distillation of the reaction mixture obtained from the tubular reactor (R), followed by
d) separation of the concentrated crude reaction mixture obtained from the distillation of step c) resulting in an alkaline aqueous phase and an organic phase containing isophorone, followed by
e) distillation of the organic phase containing isophorone recovered in the preceding step, followed by
f) distillation of the polycondensation by-products from the bottom of the column (D2) obtained from the preceding distillation, followed by
g) recycling of the stream obtained from the top of the distillation column (D3) of the preceding step comprising xylitones and/or isoxylitones to the tubular reactor (R).

### TECHNICAL FIELD

The present invention relates to a process for the continuous synthesis of isophorone by alkaline self-condensation of acetone in the liquid phase.

### TECHNICAL BACKGROUND

Isophorone (or 3,5,5-trimethylcyclohex-2-enone) is an α,β-unsaturated cyclic ketone of increasing use as a synthetic intermediate, in particular for the manufacture of isophorone diamine, which is used as a hardener for epoxy resins, isophorone diisocyanate, which is used as a polyurethane monomer, 3,5-xylenol, which is used as a precursor of PCMX (antimicrobial agent), keto-isophorone, which is a synthesis intermediate of vitamin E and 3,5,5-trimethycyclohexanol, which is used as a precursor of homosalate (UV absorber). Isophorone is also an excellent high-boiling-point solvent for many natural and synthetic resins, used in the paint, ink and varnish industries. Isophorone is also a solvent used in agrochemistry for the formulation of emulsifiable pesticide concentrates.

Isophorone is conventionally obtained by catalytic self-condensation of 3 molecules of acetone, according to the following reaction:

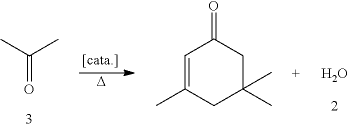

The reaction is carried out in the liquid phase or in the gas phase.

The gas-phase processes described in the literature essentially use solid heterogeneous catalysts, whereas the liquid-phase processes use homogeneous or heterogeneous catalytic systems.

The synthesis of isophorone by condensation of acetone in the liquid phase is carried out almost exclusively under alkaline conditions at high temperature and under high pressure; the alkaline catalysis being carried out most often by using an aqueous solution of sodium hydroxide or potassium hydroxide.

Owing to the low solubility of the mineral base in acetone, processes are sought that aim to promote the contact of acetone with the catalyst. Thus, it is known from document U.S. Pat. No. 2,344,226 to carry out the synthesis in a stirred reactor. Document FR 1 238 954 discloses a synthesis using a tubular reactor with internal packing. Document U.S. Pat. No. 2,399,976 discloses a synthesis using a tubular reactor equipped with a recirculation system. Documents CN 102367223 and CN 102516051 disclose a synthesis using a premixing system, such as a static mixer. It is also known from document FR 1042057 to replace the alkaline aqueous solution with an alkaline alcoholic solution.

The synthesis can be carried out continuously in a tubular reactor without special mixing equipment via the use of very low concentrations by weight of sodium hydroxide or potassium hydroxide. Generally, the catalyst concentrations are less than 1% by weight, or even of the order of 0.1% by weight relative to the total weight of the reaction mixture. This low concentration allows a single-phase mixture. These processes are described in the documents FR 1 316 515, DD145096, EP 2 649 032, EP 2 707 352 and EP 2 837 618.

The synthesis can also be carried out by reactive distillation via the injection of acetone and an aqueous solution of sodium hydroxide or potassium hydroxide into a reactive distillation column so as to maintain a low concentration of sodium hydroxide or potassium hydroxide (<0.1% by weight relative to the total weight of the reaction mixture) and to react acetone countercurrent to the sodium hydroxide or potassium hydroxide. This process is described in documents FR 1 315 788, FR 2 271 191 and FR 2 328 686.

Owing to the extreme reaction conditions used, the acetone self-condensation reaction is accompanied by the formation of polycondensation by-products formed from a number of acetone molecules of greater than or equal to 4. In order to limit the production of these heavy derivatives, the synthesis is carried out with limited acetone conversion. In addition to isophorone and the polycondensation derivatives, the crude reaction mixture also contains a greater or lesser amount of synthesis intermediates, most of which are mesityl oxide.

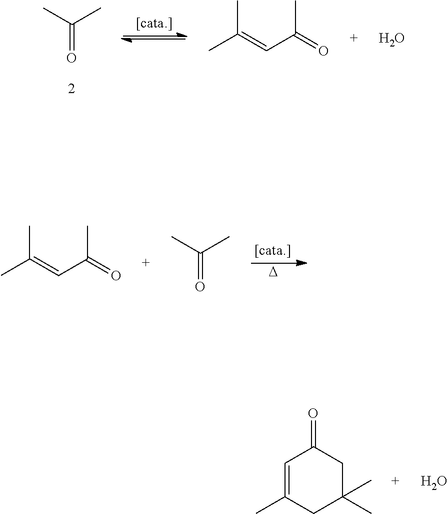

Document U.S. Pat. No. 2,344,226 describes the recycling of mesityl oxide in the reactor with the unconverted acetone. Document U.S. Pat. No. 2,351,352 describes a process in which mesityl oxide is separately reverted to acetone by hydrolysis in the presence of an alkaline aqueous solution in a reactive distillation column. In the specific case of isophorone synthesis via reactive distillation, mesityl oxide is reverted in situ to acetone (FR 1 316 515).

Document U.S. Pat. No. 2,419,051 describes the partial reversion of the polycondensation products to acetone and isophorone by alkaline hydrolysis in a stirred reactor. This reversion is also described in documents FR 1 316 514, FR 1 316 515, EP 2 649 032, EP 2 707 352 and EP 2 837 618 via a hydrolysis reactive distillation column.

The synthesis of IPHO by condensation of acetone in the gas phase is carried out at high temperature (200-400° C.) through a fixed bed of solid catalyst(s) such as for example: calcium oxide and/or calcium hydroxide (FR 850 334), a mixed magnesium-aluminium oxide catalyst (EP 0 640 387), a calcium aluminate (U.S. Pat. No. 2,393,510), zeolites or magnesium oxide doped with alkali metals (JP 9151152, JP 9151153, JP 9169687, JP 9169688)) or hydrotalcites (CN 106423124, CN 106423125, CN 106423126).

Regardless of whether a liquid-phase process or gas-phase process is used, due to the high reaction temperatures necessary for the synthesis of isophorone, the selectivity of the reaction is limited by the formation of polycondensation by-products of empirical formula C₃ₙH₍₄ₙ₊₂₎O (with n≥4), including predominantly C₁₂H₁₈O xylitones and isoxylitones and C₁₅H₂₂O compounds.

For liquid-phase syntheses, even with limited conversion, the selectivity for isophorone at the outlet of the stirred or tubular reactors is at best 75%.

Only reactive distillation synthesis processes, which are accompanied by a partial reversion of the polycondensation products in situ, and processes coupling a tubular reactor and a reactive distillation column for hydrolysis of the heavy products make it possible to achieve overall isophorone selectivities of between 85% and 91%.

However, since such reactive distillation equipment must operate under high pressures generally of between 30 and 50 bar and require specific materials capable of withstanding the alkaline conditions of the reaction medium and temperatures above 200° C. is particularly expensive.

Unlike liquid-phase processes, gas-phase processes can be operated at atmospheric pressure, but they have the major disadvantage of a decrease in reaction performance as the catalyst ages, due to fouling by the polycondensation by-products and coking caused by the use of high reaction temperatures. Industrial production is therefore greatly impacted by a frequent need to regenerate or change the catalyst bed.

There is therefore a need for a selective process, of stable productivity and that is less expensive in terms of investments than processes employing one (or more) reactive distillation(s).

### BRIEF DESCRIPTION OF THE INVENTION

The present invention relates to a continuous process for the liquid-phase synthesis of isophorone by alkaline self-condensation of acetone, comprising the following successive steps:
a) continuous injection through a tubular reactor (R) of a stream of an aqueous alkali metal hydroxide solution (A) and of a stream of an organic solution comprising acetone and by-products recycled via step g), followed by
b) condensation reaction of acetone within the tubular reactor (R), the reactor containing an emulsion predominantly comprising an aqueous alkali metal hydroxide phase (B), the concentration of alkali metal hydroxide in the aqueous phase (B) within the tubular reactor being greater than or equal to 50 g/l, followed by
c) distillation of the reaction mixture obtained from the tubular reactor (R), followed by
d) separation of the concentrated crude reaction mixture obtained from the distillation of step c) resulting in an alkaline aqueous phase and an organic phase containing isophorone,
preferably neutralization of the organic phase recovered at the end of the separation d), followed by
e) distillation of the organic phase containing isophorone recovered in the preceding step in order to extract the polycondensation by-products predominantly at the bottom of the column (D2) and to recover a stream comprising predominantly isophorone at the top of the column (D2), followed by
f) distillation of the polycondensation by-products from the bottom of the column (D2) obtained from the preceding distillation, followed by
g) recycling of the stream obtained from the top of the distillation column (D3) of the preceding step comprising xylitones and/or isoxylitones to the tubular reactor (R).

Other advantageous characteristics of the process according the invention are specified below:
the aqueous alkali metal hydroxide solution (A) is an aqueous solution of sodium hydroxide or potassium hydroxide;
the concentration of alkali metal hydroxide in the aqueous phase (B) present in the tubular reactor is between 50 and 200 g/l, preferably between 80 and 150 g/l and more preferentially between 100 and 150 g/l;
the concentration of alkali metal hydroxide in the aqueous solution (A) at the feed zone is between 5 and 40 g/l, preferably between 10 and 40 g/l and more preferentially between 15 and 35 g/l;
the ratio of the mass flow rate of the stream of aqueous alkali metal hydroxide solution (A) supplied (Qalkali metal hydroxide) to the mass flow rate of the organic stream supplied (Qorga) is between 0.25 and 1.0, preferably between 0.4 and 0.8 and more preferentially between 0.5 and 0.7
the reaction temperature within the tubular reactor is between 180° C. and 250° C., and preferably between 200° C. and 230° C., and/or the absolute pressure within the tubular reactor is between 30 and 50 bar, preferably between 35 and 45 bar, and even more preferentially between 38 and 42 bar;
the process comprises a step h) of distillation of the stream recovered at the top of the distillation column (D2) of step e);
the process comprises a step i) of distillation of the stream recovered at the bottom of the distillation column (D4) of step h);
the process comprises a step of recycling the stream recovered at the bottom of the distillation column (D5) of the preceding step to the distillation column (D2) of step e);
the process comprises a step of decantation of the stream recovered at the top of the distillation column (D4) of step h), followed by a step of recycling the organic phase to the tubular reactor (R).

The process according to the invention has the advantage of achieving levels of productivity and selectivity similar to those generally obtained with reactive distillations, but without using this type of apparatus. Specifically, the systematic recycling of fractions containing synthesis intermediates, such as mesityl oxide and fractions containing revertible by-products makes it possible to achieve these productivity and selectivity thresholds.

### BRIEF DESCRIPTION OF THE FIGURE

FIG. 1 is a scheme of the device performing the claimed process.

### DETAILED DESCRIPTION

Other characteristics, aspects, subjects and advantages of the present invention will emerge even more clearly on reading the description that follows.

It is specified that the expressions “from . . . to . . . ” and “between . . . and . . . ” used in the present description should be understood as including each of the limits mentioned.

The process according to the invention comprises the seven abovementioned consecutive steps: steps a) to g). This process may include additional purification steps.

### Step a): Injection of the Streams

The synthesis is carried out by continuous injection through a tubular reactor (R):
of a stream of an aqueous alkali metal hydroxide solution (A) and
of a stream of an organic solution comprising acetone and by-products recycled via step g).

### Aqueous Alkali Metal Hydroxide Solution (A)

The alkali metal hydroxide used is preferably sodium hydroxide or potassium hydroxide, and more preferentially sodium hydroxide in the form of an aqueous sodium hydroxide solution.

Preferably, the alkali metal hydroxide of the aqueous solution (A) is identical to the alkali metal hydroxide of the aqueous solution (B).

The concentration of alkali metal hydroxide in the reactor depends on the concentration of the aqueous alkali metal hydroxide solution (A) and on the ratio of the flow rates of the alkaline aqueous solution (A) and the organic solution at the feed zone of the reactor.

Preferably, the concentration of alkali metal hydroxide in the aqueous solution (A) at the feed zone is between 5 and 40 g/l, preferably between 10 and 40 g/l and more preferentially between 15 and 35 g/l.

If the alkali metal hydroxide is sodium hydroxide, then the concentration of sodium hydroxide in the aqueous solution at the feed zone is advantageously between 5 and 30 g/l, preferably between 10 and 30 g/l and more preferentially between 15 and 25 g/l.

If the alkaline hydroxide is potassium hydroxide, then the concentration of potassium hydroxide in the aqueous solution at the feed zone is advantageously between 5 and 40 g/l, preferably between 10 and 40 g/l and more preferentially between 20 and 35 g/l.

Preferably, the start-up of the unit is carried out by preloading the reactor with an aqueous alkali metal hydroxide solution (B).

### The Organic Solution

The organic stream comprises acetone, recycled by-products and optionally recycled reaction intermediates.

The term “recycled by-products” means polycondensation by-products that can be reverted under the isophorone synthesis conditions, namely xylitones and/or isoxylitones (C₁₂H₁₈O). Isoxylitones and xylitones comprise several isomers including the following molecules:

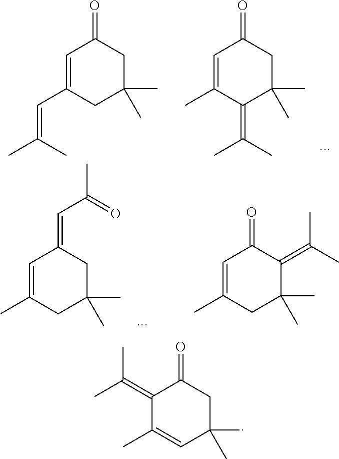

These various C₁₂H₁₈O isomers are formed by condensation of isophorone with acetone or by self-condensation of mesityl oxide:

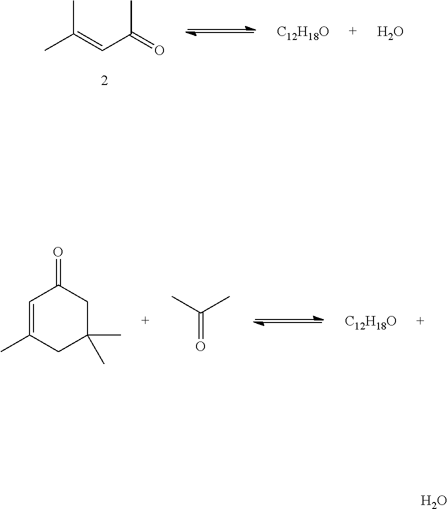

In order to optimize the selectivity for isophorone, the acetone condensation reaction is carried out with acetone conversion limited to less than 50%, preferably to less than 30% and more preferentially with an acetone conversion of between 15% and 25%.

As expanded on below, the organic stream can also comprise recycled acetone, originating from one or more distillations of the process.

In addition to the fresh acetone and recycled acetone, the organic stream may include recycled reaction intermediates, such as mesityl oxide.

The ratio of the mass flow rate of the stream of aqueous alkali metal hydroxide solution (A) supplied (Qalkali metal hydroxide) to the mass flow rate of the organic stream supplied (Qorga) is advantageously between 0.25 and 1.0, preferably between 0.4 and 0.8 and more preferentially between 0.5 and 0.7.

When the alkali metal hydroxide is sodium hydroxide, the ratio of the mass flow rate of the stream of aqueous sodium hydroxide solution supplied (QNaOH) to the mass flow rate of the organic stream supplied (Qorga) is advantageously between 0.25 and 1.0, preferably between 0.4 and 0.8 and more preferentially between 0.5 and 0.7.

When the alkali metal hydroxide is potassium hydroxide, the ratio of the mass flow rate of the stream of aqueous alkali metal hydroxide solution supplied (QKOH) to the mass flow rate of the organic stream supplied (Qorga) is advantageously between 0.25 and 1.0, preferably between 0.4 and 0.8 and more preferentially between 0.5 and 0.7.

Before entering the reactor, the streams can be preheated using heat exchangers.

### Step b): Reaction

The acetone condensation reaction is carried out within the tubular reactor (R), the reactor containing an emulsion predominantly comprising an aqueous alkali metal hydroxide phase (B), the concentration of alkali metal hydroxide in the aqueous phase (B) within the tubular reactor being greater than or equal to 50 g/l.

Preferably, the reactor is vertical.

The emulsion present within the reactor comprises:
an aqueous phase (B), which is the continuous phase of the emulsion and which comprises water, acetone and alkali metal hydroxide, and
an organic phase, which is the dispersed phase of the emulsion; it is preferably in the form of droplets and it comprises acetone, isophorone, optionally synthesis intermediates and polycondensation by-products.

The emulsion predominantly contains the aqueous alkali metal hydroxide phase. For the purposes of the present invention, the term “predominantly” means that the aqueous alkali metal hydroxide phase represents more than 50% by volume relative to the total volume of the emulsion present in the reactor.

The concentration of alkali metal hydroxide in the aqueous phase (B) within the tubular reactor is greater than 50 g/l.

In contrast to the liquid-phase processes catalyzed with sodium hydroxide or potassium hydroxide described in the literature, which employ conditions for having a reaction phase which is as homogeneous as possible, the process according to the invention carries out a reaction in a heterogeneous medium. This makes it possible to increase the selectivity for isophorone.

The heterogeneous reaction medium is formed of the continuous aqueous phase concentrated in alkali metal hydroxide through which the organic phase passes in the form of droplets, preferably ascending droplets. The much lower solubility of isophorone compared to acetone in this concentrated alkaline aqueous phase thus makes it possible to limit the formation of polycondensation by-products.

The reaction medium is thus heterogeneous. It comprises a majority aqueous alkali metal hydroxide phase and a minority organic phase comprising acetone, isophorone and optionally the recycled organic by-products, and optionally the synthesis intermediates.

The reaction temperature within the tubular reactor may be between 180° C. and 250° C., and preferably between 200° C. and 230° C., and under an absolute pressure of between 30 and 50 bar, preferably between 35 and 45 bar, and even more preferentially between 38 and 42 bar.

The concentration of alkali metal hydroxide in the aqueous phase (B) present in the tubular reactor may be between 50 and 200 g/l, preferably between 80 and 150 g/l and more preferentially between 100 and 150 g/l.

When the alkali metal hydroxide is sodium hydroxide, the concentration of sodium hydroxide in the aqueous phase (B) present within the tubular reactor is preferably between 50 and 200 g/l, preferably between 80 and 150 g/l and more preferentially between 100 and 120 g/l.

When the alkaline hydroxide is potassium hydroxide, the concentration of potassium hydroxide in the aqueous phase (B) present within the tubular reactor may be between 50 and 200 g/l, preferably between 80 and 150 g/l and more preferentially between 125 and 150 g/l.

The tubular reactor R may, where appropriate, consist of several tubular reactors fed in parallel.

The reaction mixture is recovered at the outlet of the tubular reactor and conveyed to a distillation column.

### Distillations

In the process according to the invention, the distillation columns preferably comprise a boiler at the bottom of the column and a condenser at the top of the column. The columns may be plate columns or packed columns.

Advantageously, the distillations are carried out under reduced pressure.

Distillation under reduced pressure corresponds to a distillation carried out at an absolute pressure of less than 1013 mbar, preferably less than 250 mbar and more preferentially of between 10 and 100 mbar.

Preferably, the process according to the invention does not include reactive distillation.

### Step c): Distillation 1

The reaction mixture, recovered at the reactor outlet, is distilled through a column D1. The unconverted acetone is recovered at the top of the column and the concentrated crude reaction mixture is drawn off at the bottom of the column. Preferably, the distillation is carried out at atmospheric pressure.

### Optional Additional Recycling

The acetone recovered at the top of column D1 is advantageously recycled to the tubular reactor R, completely or in part.

### Step d): Separation

The concentrated crude reaction mixture drawn off at the bottom of distillation column D1 is separated, preferably by decantation. The alkaline aqueous phase is separated from the isophorone-rich organic phase, preferably by means of a decanter.

### Optional Additional Recycling

The alkaline aqueous phase recovered in the separation step d) is advantageously recycled to reaction step b), completely or in part, and preferably in part.

### Optional Neutralization of the Organic Phase

Any alkali metal hydroxide present in the isophorone-rich organic phase recovered in the separation step d) may be neutralized. This neutralization may be carried out by any technique known to those skilled in the art, but preferably by means of a mineral acid providing a buffer effect. Preferably, phosphoric acid is used.

### Step e): Distillation 2

The isophorone-containing organic phase recovered in the separation step d), and then optionally neutralized, is distilled, preferably under reduced pressure, in order to extract the polycondensation by-products predominantly at the bottom of column D2 and to recover a stream comprising predominantly isophorone at the top of column D2.

### Step f): Distillation 3

The fraction comprising the polycondensation by-products recovered at the bottom of the column of the preceding distillation step D2 is distilled, preferably under reduced pressure. The fraction obtained from the top of distillation column D3 preferably comprises predominantly xylitones and/or isoxylitones and the fraction at the bottom of the column preferably comprises predominantly polycondensation by-products of empirical formula C₁₅H₂₂O.

The C₁₅H₂₂O polycondensation by-products comprise several isomers, including the molecules below:

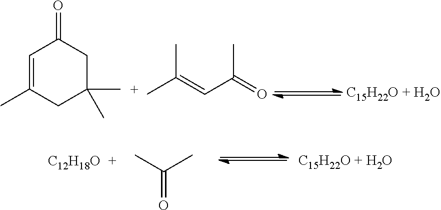

These C₁₅H₂₂O derivatives are formed by condensation of isophorone with mesityl oxide or by condensation of C₁₂H₁₈O xylitones or isoxylitones with acetone:

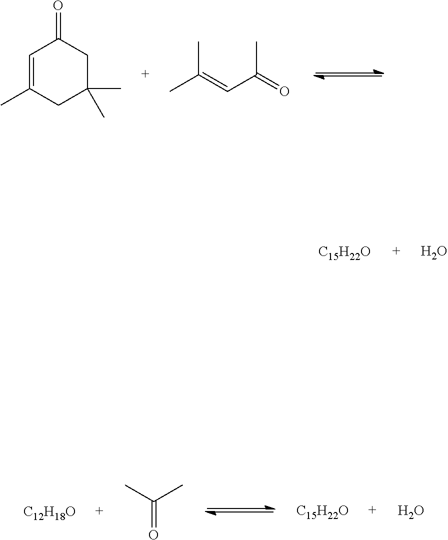

The heavy reaction by-products Si at the bottom of column D3 comprise these C₁₅H₂₂O by-products and also the higher homologues thereof C₃ₙH₍₄ₙ₊₂₎O (with n≥6).

### Step g): Recycling

The fraction obtained from the top of the column of the preceding distillation step D3, preferably comprising predominantly xylitones and/or isoxylitones, is recycled to the tubular reactor R. This fraction is added to the continuous stream of the organic phase fed into the reactor.

### Optional Decolourizing Treatment

The stream recovered at the top of column D2 may be subjected to a decolourizing treatment.

This decolourizing treatment consists in transforming certain reaction intermediates and/or by-products comprising conjugated unsaturated and carbonyl-containing hydrocarbon chains which are difficult to separate from isophorone by distillation. Their residual presence could therefore generate a yellowish colouration of the isophorone. This treatment may be carried out by any method known to those skilled in the art for oxidizing or reducing the olefinic bonds or for polycondensing the by-products held responsible.

Preferably, the decolourizing treatment comprises a step of reacting with an acid. The stream of isophorone extracted at the top of column D2 is subjected to a continuous hot treatment in the presence of a catalytic amount of a strong mineral acid, such as sulfuric acid.

The residual sulfuric acid can then be advantageously neutralized by adding a strong mineral base such as the alkaline aqueous solution, preferably the one obtained from separation step d).

### Optional Distillations 4 and 5: Steps h) and i)

The process according to the invention may comprise a step h) of distillation of the stream recovered at the top of the distillation column (D2) of step e). This stream recovered at the top of column (D2) can be sent to a subsequent distillation column, optionally after a decolourizing step.

The stream, predominantly comprising isophorone and recovered at the top of distillation column D2, may undergo a distillation, preferably under reduced pressure, in a column D4 enabling the light impurities, such as residual acetone and water, mesityl oxide and 1,3,5-trimethylbenzene to be extracted at the top of the column.

The stream extracted at the top of column D4 can be decanted through the decanter in order to separate an aqueous phase, which is sent to a wastewater treatment SE and an organic phase predominantly comprising mesityl oxide and isophorone.

This organic phase predominantly comprising mesityl oxide and isophorone can be recycled to the reaction step.

The process according to the invention may comprise a step i) of distillation of the stream recovered at the bottom of the distillation column (D4) of step h). The fraction recovered at the bottom of column D4 can feed a fifth distillation column, which makes it possible to obtain isophorone with a purity of greater than 99% at the top of the column and residual polycondensation by-products at the bottom of the column.

These residual polycondensation by-products can be recycled to distillation column D2.

### DESCRIPTION OF THE FIGURE

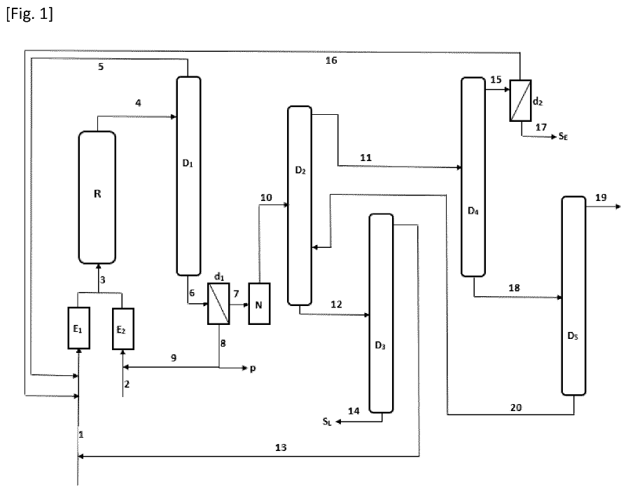

FIG. 1 represents one embodiment of steps a) to g) of the process according to the invention.

Acetone is introduced via line 1 into heat exchanger E1. The aqueous alkali metal hydroxide solution is introduced via line 2 into heat exchanger E2. The preheated streams are recovered in a line 3 and introduced into the tubular reactor R.

The reaction mixture obtained from the reactor R is introduced into the distillation column D1 via line 4.

The acetone which has not been converted in the reactor is recovered at the top of column D1. This fraction is recycled via the line 5 to the line 1.

The concentrated crude reaction mixture is recovered at the bottom of column D1 and is conveyed to the decanter d1 via line 6.

The decanter d1 separates the aqueous phase from the organic phase. The alkaline aqueous phase is removed via the line 8 and then the line 9. A purge p is introduced to remove the water co-produced by the condensation reaction. The line 9 recycles the alkaline aqueous phase to the line 2.

The organic phase obtained from the decanter d1 is conveyed to the neutralizer N via the line 7. The neutralized organic phase is conveyed to the distillation column D2 via line 10.

The distillation under reduced pressure using the distillation column D2 makes it possible to recover at the top of column D2 a stream comprising predominantly isophorone, which is transferred via the line 11 to the distillation column D4. At the bottom of column D2, the recovered fraction containing the polycondensation by-products is transferred via line 12 to the distillation column D3.

The distillation under reduced pressure of the polycondensation by-products within the distillation column D3 makes it possible to recover at the top of the column D3 a stream preferably comprising xylitones and/or isoxylitones. This fraction is recycled via the line 13 to the line 1.

The heavy reaction by-products S are recovered at the bottom of column D3.

The stream recovered at the top of column D2 and comprising predominantly isophorone, feeds distillation column D4. The distillation under reduced pressure in column D4 enables the light impurities, such as residual acetone and water, mesityl oxide and 1,3,5-trimethylbenzene to be extracted at the top of the column. This fraction is discharged via the line 15 to the decanter d2. The aqueous phase obtained from the decanter d2 is sent via the line 17 to a wastewater treatment SE and the organic phase comprising predominantly mesityl oxide and isophorone is recycled via the line 16 to the line 1.

The fraction recovered at the bottom of column D4 feeds column D5 via the line 18. The fraction recovered at the top of column D5 contains isophorone with a purity of greater than 99%. The fraction drawn off at the bottom of column D5 comprises residual polycondensation by-products, which are recycled via the line 20 into distillation column D2.

Therefore, the organic phase stream fed to the reactor R contains fresh acetone, the light fraction from distillation column D1 and recycled via line 5, the light fraction from distillation column D3 and recycled via line 13 and the organic phase obtained from the decanter d2 and recycled via line 16.

The alkaline aqueous solution (A) feeding the reactor R contains a fresh aqueous alkali metal hydroxide solution and the aqueous phase obtained from the decanter d1 and recycled via the line 9.

The examples that follow illustrate the present invention but are not in any way limiting.

### EXAMPLES

### Example 1

The synthesis of isophorone is carried out in a vertical tubular reactor made of 316 L stainless steel with a volume of 815 ml and an L/D ratio of 3, equipped with a sidearm positioned at L/2 for withdrawing a sample at the middle of the reactor.

The alkaline aqueous phase and the organic phase consisting of fresh acetone, recycled acetone and, where appropriate, recycled mesityl oxide and recycled polycondensation products are respectively preheated through 2 electric heat exchangers so as to reach the reaction temperature desired within the reactor.

The tubular reactor is prefilled with an aqueous sodium hydroxide solution, also preheated, with a sodium hydroxide concentration of 10% by weight.

The feeding of reagents is carried out by means of piston pumps.

The conversions and selectivities are established after a continuous operation of at least 24 h in order to guarantee stabilized reaction conditions within the reactor.

Table 1 below indicates the operating conditions of the tests carried out under an absolute pressure of 40 bar in the tubular reactor with a sodium hydroxide concentration of 20 g/l in the aqueous solution supplied.

In Tables 1 and 2 below, MO stands for mesityl oxide; C₁₂ denotes C₁₂reaction by-products, i.e. xylitones and/or isoxylitones; C₁₅ denotes C₁₅reaction by-products, such as C₁₅H₂₂O; C₁₈ denotes C₁₈ reaction by-products; ACE stands for acetone; IPHO stands for isophorone and the selectivities are defined as follows:

$${S}_{P} = selectivity for product P relative to the acetone converted$$

$${S}_{IPHO} = 100 \times 3 \times ( number of moles of IPHO formed ) / number of moles of ACE converted$$

$${S}_{MO} = 100 \times 2 \times ( number of moles of MO formed ) / number of moles of ACE converted$$

$${SC}_{12} = 100 \times 4 \times ( number of moles of {C}_{12} {H}_{18} O formed ) / number of moles of ACE converted$$

$${SC}_{15} = 100 \times 5 \times ( number of moles of {C}_{15} {H}_{22} O formed ) / number of moles of ACE converted$$

$${SC}_{18} = 100 \times 6 \times ( number of moles of {C}_{18} {H}_{26} O formed ) / number of moles of ACE converted$$

$$with : number of moles of P formed = ( number of moles of P at reactor outlet - number of moles of P recycled to reactor feed )$$

The conversions and selectivities are calculated on the basis of the compositions by weight of the crude mixtures at the outlet of the reaction zone; the compositions are determined by gas chromatography analyses.

TABLE 1

| | Flow rates of recycled | | | Q_{NaOH} _{aq}/ | Average | C_{NaOH} of |
|---|---|---|---|---|---|---|
| | products | | | Q_{Orga} | reaction | reactor |
| | MO_{αβ+} | C_{12 total} | C_{15 total} | weight | temperature | medium |
| | (g/h) | (g/h) | (g/h) | ratio | ° C. | (g/l) |
| 1 comp | — | — | — | 0.66 | 216 | 122 |
| 2 comp | 10.6 | — | — | 0.68 | 216 | 122 |
| 3 | 0.5 | 19.2 | 5.5 | 0.73 | 216 | 93 |
| 4 | 11.1 | 35.6 | 10.3 | 0.67 | 215 | 102 |

Test 1 is a comparative test, it illustrates a process without recycling.

Test 2 is also a comparative test, it recycles only the fractions containing mesityl oxide.

Test 3 is according to the invention, it recycles only the fractions containing xylitones and/or isoxylitones.

Test 4 is according to the invention, it recycles all the fractions containing mesityl oxide and xylitones and/or isoxylitones.

Tests 3 and 4 use a succession of 5 distillation columns and they recycle the fractions containing C₁₂ impurities, which are revertible and the C₁₅ impurities.

Table 2 below indicates the results obtained.

TABLE 2

| | | Selectivities/ACE | | | | | IPHO |
|---|---|---|---|---|---|---|---|
| | ACE conversion | IPHO | MO_{αβ+} | C_{12 total} | C_{15 total} | C_{18 total} | productivity |
| | (%) | (%) | (%) | (%) | (%) | (%) | (kg · h⁻¹ · L⁻¹) |
| 1 comp | 19.3 | 74.4 | 11.0 | 6.1 | 7.3 | 1.1 | 0.086 |
| 2 comp | 17.1 | 81.4 | 0.9 | 7.2 | 8.9 | 1.4 | 0.086 |
| 3 | 24.0 | 80.4 | 7.2 | 0.0 | 9.9 | 2.5 | 0.115 |
| 4 | 18.9 | 85.6 | -0.5 | 0.9 | 12.6 | 1.0 | 0.091 |

The results of selectivities for mesityl oxide (MO) and for xylitones and/or isoxylitones (C_{12 total}) make it possible to observe that the recycling of the mesityl oxide and of the xylitones and/or isoxylitones can lead to complete suppression of the respective production thereof.

Example 3 shows that the recycling of the single fraction containing xylitones and/or isoxylitones (C_{12 total}) enables an increase in the selectivity for isophorone, an increase in the conversion rate and an increase in productivity.

Example 4 shows that the selectivity for isophorone (IPHO) of 74.4% without recycling thus increases to 85.6% with recycling of mesityl oxide (MO) and xylitones and/or isoxylitones (C_{12 total}).

The negative selectivity for mesityl oxide expresses the fact that the amount of mesityl oxide at the outlet of the tubular reactor is lower than that at the inlet; this indicates that recycling not only prevented the formation of mesityl oxide, but also made it possible to revert the excess recycled product relative to the reaction equilibrium.

These tests show the decisive impact of the recycling of mesityl oxide and C₁₂H₁₈O xylitones and isoxylitones on the isophorone selectivity and productivity.

### Example 2

The procedure followed is similar to that of Example 1 but using a vertical tubular reactor made of 316 L stainless steel with a volume of 940 ml and an L/D ratio of 18.5, equipped with 3 sidearms positioned at L/3, L/2 and 2L/3 for withdrawing samples at one third of the length of the reactor, at the middle of the reactor and at two thirds of the length of the reactor.

Table 3 below indicates the operating conditions of the tests:

TABLE 3

| | | | | | Average | C_{NaOH} |
|---|---|---|---|---|---|---|
| | Flow rates of recycled products | | | | reaction | middle (top) |
| | MO_{αβ+} | C_{12 total} | C_{15 total} | Q_{NaOH} _{aq}/Q_{Orga} | temperature | of reactor |
| | (g/h) | (g/h) | (g/h) | weight ratio | ° C. | (g/l) |
| 5 comp | — | — | — | 0.68 | 217 | 118 (118) |
| 6 | 24.2 | 34.0 | 4.0 | 0.68 | 216 | 106 (112) |

Test 5 is a comparative test, it illustrates a process without recycling.

Test 6 is according to the invention. The process followed is that illustrated in FIG. 1, it uses a succession of 5 distillation columns and it recycles the fractions containing C₁₂ impurities, which are revertible and the C₁₅ impurities.

Table 4 below indicates the results obtained.

TABLE 4

| | | Selectivities/ACE | | | | | IPHO |
|---|---|---|---|---|---|---|---|
| | ACE conversion | IPHO | MO_{αβ+} | C_{12 total} | C_{15 total} | C_{18 total} | productivity |
| | (%) | (%) | (%) | (%) | (%) | (%) | (kg · h⁻¹ · L⁻¹) |
| 5 comp | 19.8 | 75.3 | 10.8 | 6.5 | 7.1 | 0.3 | 0.133 |
| 6 | 17.4 | 93.0 | -4.6 | -3.5 | 6.8 | 0.2 | 0.137 |

As in Example 1, a sufficiently substantial recycling of the xylitones and/or isoxylitones makes it possible to completely eliminate the formation thereof. It should be noted that the negative selectivities for mesityl oxide and for C₁₂H₁₈O express the fact that the amounts of mesityl oxide and of C₁₂H₁₈O at the outlet of the tubular reactor are lower than those at the inlet; this indicates that recycling not only prevented the formation of mesityl oxide and C₁₂H₁₈O, but also made it possible to revert the excess recycled products relative to the reaction equilibrium.

In addition, it should be noted that these recycling operations do not increase the formation of the higher C₁₈H₂₆O heavy condensation by-products.

The selectivity for isophorone of 75.3% without recycling thus increases to 93.0% with recycling of mesityl oxide (MO) and xylitones and/or isoxylitones (C_{12 total})

The saving in terms of acetone consumed under the conditions of test 6 compared with the acetone consumed under the conditions of test 5 is thus 0.32 kg of acetone per kg of isophorone produced.

1. Continuous process for the liquid-phase synthesis of isophorone by alkaline self-condensation of acetone, comprising the following successive steps:
a) continuous injection through a tubular reactor (R):
of a stream of an aqueous alkali metal hydroxide solution (A), and
of a stream of an organic solution comprising acetone and by-products recycled via step g), followed by
b) condensation reaction of acetone within the tubular reactor (R), the reactor containing an emulsion predominantly comprising an aqueous alkali metal hydroxide phase (B), the concentration of alkali metal hydroxide in the aqueous phase (B) within the tubular reactor being greater than or equal to 50 g/l, followed by
c) distillation of the reaction mixture obtained from the tubular reactor (R), followed by
d) separation of the concentrated crude reaction mixture obtained from the distillation of step c) resulting in an alkaline aqueous phase and an organic phase containing isophorone, preferably neutralization of the organic phase recovered at the end of the separation d), followed by
e) distillation of the organic phase containing isophorone recovered in the preceding step in order to extract the polycondensation by-products predominantly at the bottom of the column (D2) and to recover a stream comprising predominantly isophorone at the top of the column (D2), followed by
f) distillation of the polycondensation by-products from the bottom of the column (D2) obtained from the preceding distillation, followed by
g) recycling of the stream obtained from the top of the distillation column (D3) of the preceding step comprising xylitones and/or isoxylitones to the tubular reactor (R).

2. Process according to claim 1, characterized in that the aqueous alkali metal hydroxide solution (A) is an aqueous solution of sodium hydroxide or potassium hydroxide.

3. Process according to claim 1, characterized in that the concentration of alkali metal hydroxide in the aqueous phase (B) present within the tubular reactor is between 50 and 200 g/l, preferably between 80 and 150 g/l and more preferentially between 100 and 150 g/l.

4. Process according to claim 1, characterized in that the concentration of alkali metal hydroxide in the aqueous solution (A) at the feed zone is between 5 and 40 g/l, preferably between 10 and 40 g/l and more preferentially between 15 and 35 g/l.

5. Process according to claim 1, characterized in that the ratio of the mass flow rate of the stream of aqueous alkali metal hydroxide solution (A) supplied (Qalkali metal hydroxide) to the mass flow rate of the organic stream supplied (Qorga) is between 0.25 and 1.0, preferably between 0.4 and 0.8 and more preferentially between 0.5 and 0.7.

6. Process according to claim 1, characterized in that the reaction temperature within the tubular reactor is between 180° C. and 250° C., and preferably between 200° C. and 230° C., and the absolute pressure within the tubular reactor is between 30 and 50 bar, preferably between 35 and 45 bar, and even more preferentially between 38 and 42 bar.

7. Process according to claim 1, characterized in that it comprises a step h) of distillation of the stream recovered at the top of the distillation column (D2) of step e).

8. Process according to claim 7, characterized in that it comprises a step i) of distillation of the stream recovered at the bottom of the distillation column (D4) of step h).

9. Process according to claim 8, characterized in that it comprises a step of recycling the stream recovered at the bottom of the distillation column (D5) of the preceding step to the distillation column (D2) of step e).

10. Process according to claim 7, characterized in that it comprises a step of decantation of the stream recovered at the top of the distillation column (D4) of step h), followed by a step of recycling the organic phase to the tubular reactor (R).
